(19) United States
(12) Patent Application Publication
(10) Pub. No.: US 2020/0069837 A1
(43) Pub. Date: Mar. 5, 2020

(54) MATRIX COMPRISING BIOACTIVE GLASS

(71) Applicant: ARTERIOCYTE MEDICAL SYSTEMS, INC., St. Louis, MO (US)

(72) Inventors: Huston Davis Adkisson, IV, St. Louis, MO (US); Anthony J. Ward, St. Louis, MO (US); Brian Barnes, Upton, MA (US)

(21) Appl. No.: 16/559,219

(22) Filed: Sep. 3, 2019

Related U.S. Application Data
(60) Provisional application No. 62/725,865, filed on Aug. 31, 2018.

(51) Int. Cl. A61L 27/02; A61L 27/58; A61L 27/56; A61L 27/54; A61L 27/04; A61L 27/44; A61L 27/12; A61L 27/10; A61L 27/18

(52) U.S. Cl. CPC A61L 27/025; A61L 2300/412; A61L 27/56; A61L 27/54; A61L 27/047; A61L 27/446; A61L 27/12; A61L 27/10; A61L 27/18; A61L 2430/02; A61L 2430/30; A61L 2430/34; A61L 2300/112; A61L 2300/102; A61L 2300/10; A61L 27/58

(57) ABSTRACT

The present disclosure provides matrix compositions comprising bioactive glass and methods for treating a defect in tissue demonstrating volumetric tissue loss arising from injury or congenital defect.

### CROSS-REFERENCE TO RELATED APPLICATIONS

This application claims the benefit of U.S. Provisional Patent Application No. 62/725,865 filed Aug. 31, 2018, the entire disclosure of which is incorporated herein by reference.

### FIELD OF THE INVENTION

The present disclosure provides compositions and methods for treating a defect in tissue demonstrating volumetric tissue loss.

### BACKGROUND

Treatment of volumetric tissue loss remains a significant clinical challenge where injury to or pathologic dysfunction of connective tissue can lead to functional deficit requiring limb amputation. For instance, restoration of large segmental bone defects remains a significant clinical challenge. Historically, long bone defects greater than 5 cm in length have been treated either with autologous vascularized bone transfer or mechanical bone transfer. However, each of these surgical approaches presents a unique set of associated co-morbidities and risk for complication. Failure to salvage the pathologic limb can therefore result in unwanted amputation. Investigators have also explored the utility of polymer sheets or titanium cages that offer no biologic advantage to bone regeneration, despite providing excellent containment of bone graft.

Further, the Masquelet technique, commonly utilized to generate a vascularized matrix for graft containment and bone induction, has been employed successfully for nearly three decades. However, the technique is both technically demanding and subjects patients to multiple surgical procedures with a protracted period of non-weight bearing—typically greater than 5 months. The technique requires a two-staged surgical procedure whereby a soft-tissue envelope is first created through a foreign body reaction for secondary placement of bone graft at approximately two months from initial surgery.

Therefore, there remains a great unmet medical need for compositions and methods that aid in treating defects in tissue demonstrating volumetric tissue loss, including regeneration of musculoskeletal tissue such as large segmental bone defects, which compositions and methods are capable of simplifying procedures used for treating such defects, and shortening the time required for tissue healing.

### SUMMARY OF THE INVENTION

One aspect of the present disclosure encompasses a matrix composition for treating a defect in tissue demonstrating volumetric tissue loss. The matrix comprises a polymer membrane comprising a first surface and a second surface, and bioactive glass associated with a surface of the polymer membrane. The bioactive glass comprises an inorganic element capable of facilitating tissue healing.

The matrix can be biocompatible, flexible, resorbable, or combinations thereof. The bioactive glass can be in the form of fibers or spheres and can be porous. The bioactive glass can be borate glass comprising about 50-55 wt % borate, about 0% silicate, and about 3.0-5.0% wt phosphate.

The inorganic element can be selected from Cu, Se, Co, Zn, Li, and combinations thereof. In some aspects, the inorganic element can be Cu, Zn, and Li, Cu and Li, or Co and Li. The bioactive glass can be associated with the first surface of the polymer membrane.

The polymer membrane can further comprise a therapeutic concentration of the inorganic element embedded within the polymer membrane. The inorganic element embedded within the polymer membrane can be formulated in the form of beads made from alginate, collagen or dextran, glass, or silicate.

In some aspects, the polymer membrane comprises an internal polymer layer and at least one polymer layer in contact with each surface of the internal polymer layer, wherein the internal polymer layer further comprises a therapeutic concentration of the inorganic element embedded within the internal polymer layer, and wherein the bioactive glass is associated with at least one surface of the polymer membrane.

Another aspect of the present disclosure encompasses a method of treating a defect in tissue demonstrating volumetric tissue loss. The method comprises obtaining the matrix described in this section above, contacting healthy tissue neighboring the tissue loss with the matrix, and surrounding the defect with the matrix thereby forming an enclosure around the tissue loss.

The tissue loss can be segmental bone loss. When the tissue loss is segmental bone loss, the method can further comprise containing bone graft material within the enclosure. The bone graft material can comprise patient derived bone marrow aspirate concentrate.

The volumetric tissue loss can also be muscular tissue loss. When the volumetric tissue loss is muscular tissue loss, the method can further comprise containing muscle graft material within the enclosure. The muscle graft material can comprise viable muscle tissue, lipoaspirate, and microvascular fragments. In some aspects, the defect is connective tissue loss.

Yet another aspect of the present disclosure encompasses a method of manufacturing the matrix described above. The method comprises obtaining the bioactive glass, obtaining a polymer membrane, and associating the bioactive glass with a layer of polymer membrane, thereby forming the matrix.

### BRIEF DESCRIPTION OF THE DRAWINGS

FIG. 1 is a representation of a matrix composition comprising a layer of bioglass (top layer) associated with one surface of a resorbable polymer membrane layer (bottom layer).

FIG. 2 shows a representation of a matrix composition comprising a single resorbable polymer membrane layer (middle layer) and a layer of bioglass (top and bottom layers) associated with each surface of polymer membrane layer.

FIG. 3 is a representation of a matrix composition comprising a single internal resorbable polymer membrane layer (from top: third layer), two external resorbable polymer membrane layers (from top: second and fourth layers) each associated with a surface of the internal membrane, and a layer of bioglass (from top: first and fifth layer) associated with each external surface of each external polymer membrane layer.

FIG. 4 is a representation of a matrix composition comprising a single internal resorbable polymer membrane layer (from top: second layer), two external resorbable polymer membrane layers (from top: first and third layers) each associated with a surface of the internal membrane, and a single layer of bioglass (from top: fourth layer) associated with an external surface of one of the external polymer membrane layers.

FIG. 5 shows a representation of a matrix composition wherein a polymer membrane further comprises inorganic elements in the form of resorbable beads (dots).

### DETAILED DESCRIPTION

The present disclosure encompasses compositions and methods for treating a defect in tissue demonstrating volumetric tissue loss. The defect can arise from injury or congenital defect. The compositions and methods comprise a matrix composition created through the combination of a resorbable polymer membrane, bioactive glass, and inorganic elements. Importantly, a matrix composition is capable of providing localized and timed delivery of inorganic elements to the tissue to promote rapid vascularization and restoration of tissue. While not wishing to be bound by theory, it is believed that tissue repair recapitulates embryonic tissue development through LRP5-independent WNT signaling, a process recognized to deteriorate with age in humans and other mammals.

Significantly, the compositions are capable of regenerating lost tissue and shortening the time required for healing the tissue, including bone healing and pain-free weight bearing. The compositions also limit the frequency and severity of the surgical procedures used to repair the tissue, and obviate the need for administration of antibiotics during the surgical procedures.

### I. Compositions

One aspect of the disclosure comprises a matrix composition for treating a defect in tissue demonstrating volumetric tissue loss. The composition comprises a polymer membrane comprising a first surface and a second surface, and bioactive glass associated with a surface of the polymer membrane. The bioactive glass comprises an inorganic element capable of facilitating tissue healing.

(a) Bioactive Glass

As used herein, the terms “bioactive glass” and “bioglass” are used interchangeably, and refer to a glass composition which, when contacted with living tissue, induces biological activity in the living tissue. For instance, when a bioactive glass of the disclosure is contacted with bone tissue, the bioactive glass induces biological activity that results in healing and restoration of injured tissue. It will be recognized that bioactive glass is also biocompatible to minimize reaction when contacted with tissue. Further, bioactive glass of the disclosure can be biodegradable.

The bioactive glass can be of any size and shape, provided the glass supplies the desired bioactive characteristics. For instance, the size and shape of bioactive glass can and will vary depending on the intended tissue to be repaired, the intended procedure used for repairing volumetric tissue loss, and the membrane composition with which the bioactive glass is associated, among other factors, and can be determined experimentally using methods recognized in the art. For instance, the bioactive glass can be spherical, cylindrical, conical, cubicle, or fibrous. In some aspects, the bioactive glass is in the form of fibers. In other aspects, the bioactive glass is in the form of spheres. Further, the bioglass can be solid or porous.

Bioactive glass and methods of preparing bioactive glass are known in the art and can be as disclosed in, e.g., U.S. Pat. No. 8,337,875, U.S. Patent Publication No. 2009/0208428, U.S. Patent Publication No. 2006/0233887, and U.S. Pat. No. 6,709,744, the disclosures of which are incorporated herein in their entirety. In some aspects, a biodegradable bioactive glass of the disclosure can be as disclosed in U.S. Pat. No. 8,337,875. In one aspect, a bioactive glass of the disclosure is a borate glass comprising the glass formers borate (B₂O₃), silicate (SiO₂), and phosphate (P₂O₅).

Biodegradability of bioactive glass of the disclosure can be tuned to provide a desired rate of dissolution and tissue residence time. Further, the desired rate of dissolution and tissue residence time of bioactive glass can and will vary depending on the intended tissue to be repaired, the intended procedure used to be repaired, and the membrane composition with which the bioactive glass is associated, among other factors, and can be determined experimentally using methods recognized in the art.

Residence time of bioglass, when a composition of the disclosure is contacted with tissue, can range from about 1 day to about 200 days, from about 10 to about 100 days, or from about 15 to about 50 days. The residence time of bioglass can range from about 20 days to about 40 days when a composition of the disclosure is contacted with tissue.

When a composition of the disclosure is contacted with tissue, time to complete dissolution can range from about 1 day to about 2 years, from about 1 day to about 1 year, from about 1 day to about 1 week, from about 1 day to about 1, 2, 3, 4, 5, 6, 7, 8, 9, or 10 months, from about 1 week to about 1 year, or from about 1 month to about 1 year.

Desired biodegradability of bioactive glass can be tuned by balancing the concentrations of glass formers used to prepare a bioactive glass of the disclosure. For instance, when the bioactive glass is a borate glass comprising borate (B₂O₃), silicate (SiO₂), and phosphate (P₂O₅), biodegradability of bioactive glass may be tuned by balancing the concentrations of the glass formers with respect to each other and with respect to other components in the glass. The concentration of B₂O₃ can range from about 40-80%, from about 40-60%, or from about 45-58% with respect to other components in the glass. The concentration of SiO₂ can range from about 0-20%, from about 0-10%, or from about 0-5% with respect to other components in the glass. The concentration of P₂O₅ can range from about 0-20%, from about 0-10%, or from about 0-5% with respect to other components in the glass. In one aspect, the bioactive glass comprises about 50-55 wt % borate, about 0% silicate, and about 3.0-5.0 wt % phosphate, with respect to other components in the glass material.

Bioactive glass of the disclosure comprises therapeutic concentrations of inorganic elements. Any inorganic element capable of facilitating tissue healing can be suitable for a composition of the disclosure. Non-limiting examples of inorganic elements capable of facilitating tissue healing include B, Cu, F, Fe, Mn, Mo, Ni, Si, Co, Se, Sr, Zn, Li, and combinations thereof. In some aspects, an inorganic element can be selected from Cu, Se, Co, Zn, Li, and combinations thereof. In some aspects, an inorganic element is selected from Cu, Zn, and Li, Cu and Li, or Co and Li.

Inorganic elements can be in any form suitable for facilitating tissue healing. For instance, inorganic elements can be ions or salts of the elements, oxides of the elements, or inorganic elements complexed with other compounds or molecules such as chelators, proteins, or peptides. Preferred forms of inorganic elements are ionic lithium or a lithium salt such as LiCl₂, a sulfate salt of zinc, an oxide of zinc such as ZnO or other Zn compounds such as Zn3(PO4)2−xH2O, copper sulfate, copper nitrate, a copper oxide such as CuO or Cu2O, a cobalt oxide, SrO, and SrCO3.

Therapeutic concentrations of each inorganic element in bioactive glass can and will vary depending on the bioactive glass and/or on the composition and the intended use of the composition, among other variables, and can be determined experimentally. In general, inorganic elements can be incorporated into bioactive glass in a concentration ranging from about 0.05% w/w to about 10% w/w or more. For instance, when an inorganic element is Cu, the Cu can be a copper oxide such as CuO or Cu₂O or other copper compounds such as copper nitrate or copper sulfate, for example. In some aspects, the concentration of Cu in the bioactive glass can range between about 0.05 and about 5 wt % (about 0.06-6 wt % CuO; about 0.055-5.5 wt % Cu₂O), or between about 0.1 and about 2.5 wt % (about 0.12-3 wt % CuO; about 0.11-3 wt % Cu₂O). In some aspects, the concentration of Cu in the bioactive glass ranges from about 1 wt % to about 2 wt % Cu.

When an inorganic element is Sr, the Sr can be an oxide such as SrO or other Sr compounds such as SrCO₃, for example. In some aspects, the concentration of Sr in the bioactive glass can range between about 0.05 and about 5 wt % (about 0.06 to 5.90 wt % SrO), or between about 0.1 and about 2.5 wt % (about 0.12 to 2.95 wt % SrO). In some aspects, the concentration of Sr in the bioactive glass ranges from about 1 wt % to about 3 wt % Sr.

When an inorganic element is Zn, the Zn can be an oxide such as ZnO or other Zn compounds such as Zn₃(PO₄)₂₋ₓH₂O, for example. In some aspects, the concentration of Zn in the bioactive glass can range between about 0.05 and about 5 wt % (about 0.06 to 6.0 wt % ZnO), or between about 0.1 and about 2.5 wt % (about 0.12 to 3.0 wt % ZnO). In some aspects, the concentration of Zn in the bioactive glass ranges from about 1 wt % to about 2 wt % Zn, or from about 1 wt % to about 3 wt % ZnO.

When an inorganic element is Fe, the Fe can be an oxide such as FeO, Fe₃O₄, Fe₂O₃, or other Fe compounds such as FeSO₄-7H₂O, for example. In some aspects, the concentration of Fe n the bioactive glass can range between about 0.05 and about 5 wt % (about 0.06 to 6.45 wt % FeO), or between about 0.1 and about 2.5 wt % (about 0.13 to 3.23 wt % FeO). In some aspects, the concentration of Fe in the bioactive glass ranges from about 1 wt % to about 2 wt % Fe, or from about 1 wt % and about 3 wt % FeO.

(b) Polymer Membrane

A polymer membrane of the composition generally comprises biocompatible polymers. The biocompatible polymers can be resorbable. Resorbable biocompatible polymers suitable for a membrane of the disclosure are known in the art. See, e.g., Shimp, N. G. (2018) “Biodegradable and Biocompatible Polymer Composites; Processing, Properties and Applications,” Elsevier Science, the disclosure of which is incorporated herein in its entirety. A polymer membrane of the composition can be flexible. Flexible resorbable membrane material can be as disclosed in U.S. application Ser. No. 14/510,917, the disclosure of which is incorporated herein in its entirety.

In some aspects, resorbable biocompatible polymers suitable for a membrane of the disclosure comprise polyester polymer molecules. Non-limiting examples of polyester polymers suitable for a membrane of the disclosure include polylactic acid (PLA); polyglycolic acid (PGA); polycaprolactone (PCL); polyethylene glycol; a copolymer comprising PLA and PGA (also referred to as poly(lactide-co-glycolide), PLA-PGA, or PLGA; a co-polymer of polylactic acid (PLA) and polycaprolactone (poly(lactide-co-caprolactone) (PLCL); a co-polymer of polyglycolic acid (PGA) and caprolactone (poly(glycolide-co-caprolactone) (PGCL); a co-polymer of polycaprolactone and both polylactic acid and polyglycolic acid (e.g., PGA-PLCL, PLA-PGCL); a co-polymer of polyethylene glycol (PEG), polylactic acid and polycaprolactone (e.g., PEG-PLCL, PLA-PEG-PCL and PLA-PEG-PLCL); a co-polymer of polyethylene glycol, polyglycolic acid and polycaprolactone (e.g., PEG-PGCL, PGA-PEG-PCL and PGA-PEG-PGCL); a co-polymer of polyethylene glycol, polylactic acid, polyglycolic acid, and polycaprolactone (e.g., PLA-PEG-PGCL, PGA-PEG-PLCL, PLA-PEG-PGA-PCL; PGA-PEG-PLA-PCL), polyhdroxyalkanoates e.g. Poly (4-hydroxybutyric acid), and combinations thereof. Preferred polyester polymers are PLGA, PGCL, and combinations thereof. In some aspects, a resorbable biocompatible polymer is polyhdroxyalkanoates e.g. Poly (4-hydroxybutyric acid).

The arrangement of polymer and/or co-polymer chains, also referred to as polymer architecture, can be linear, branched, and can further be crosslinked. Branched polymers and/or co-polymers comprise a single main chain with one or more polymeric side chains, and can be grafted, star-shaped or have other architectures. When a polymer is a co-polymer, the copolymer can comprise alternating copolymers, statistical copolymers, and block copolymers. Further, when a polymer is a co-polymer, the component polymer acids can be in any weight ratio suitable for the membrane. A co-polymer can be obtained from a commercial supplier, or can be prepared according to well-known techniques, as described in references such as, in non-limiting example, Fukuzaki, Biomaterials 11: 441-446, 1990, and Jalil, J., Microencapsulation 7: 297-325, 1990, the disclosures of which are incorporated herein in their entirety.

Physical characteristics of a polymer in a membrane, such as flexibility, adsorption rate, or tissue residence time, can be adjusted by adjusting the composition and arrangement of the polymer. For instance, physical characteristics of the polymer molecules can be adjusted by adjusting the molecular weight of the polymer, the ratio and arrangement of the structural units along the polymer chain, and arrangement of the polymer chain(s) of the polymer. For instance, if the polymer is poly (lactide-co-glycolide), physical characteristics of the polymer can be adjusted by adjusting the molecular weight, the ratio of glycolide/lactide in the polymer, the arrangement of the poly (lactide-co-glycolide) polymer chains, and combinations thereof.

Biodegradability of a polymer membrane of the disclosure can be tuned to provide a desired rate of dissolution and tissue residence time. A desired rate of dissolution and tissue residence time of polymers can and will vary depending on the intended tissue to be repaired, the intended procedure used to be repaired, and the membrane composition with which the bioactive glass is associated, among other factors, and can be determined experimentally using methods recognized in the art. Residence time of a polymer membrane, when a composition of the disclosure is contacted with tissue, can range from about 1 day to about 200 days, from about 10 to about 100 days, or from about 15 to about 50 days. In some aspects, the residence time of a polymer membrane can range from about 20 days to about 40 days when a composition of the disclosure is contacted with tissue. Also preferred, the residence time of a polymer membrane can range from about 30 days to about 60 days when a composition of the disclosure is contacted with tissue.

Rate of dissolution of a polymer membrane when a composition of the disclosure is contacted with tissue, can range from about 1 day to about 2 years, from about 1 day to about 1 year, from about 1 day to about 1 week, from about 1 day to about 1, 2, 3, 4, 5, 6, 7, 8, 9, or 10 months, from about 1 week to about 1 year, or from about 2 weeks to about 1 year.

A polymer membrane can further comprise additional components that can influence the performance of a composition of the disclosure. For instance, a polymer membrane can further comprise components having desirable therapeutic characteristics for use with the composition (FIG. 5). Including these components in the matrix can provide timed release of said components as the membrane degrades when contacted with tissue.

The additional components can be dispersed throughout a polymer membrane. For instance, additional components can be formulated in an aqueous solution and combined with polymers in an emulsion, wherein timed dissolution of the polymer membrane provides timed release of said components. Alternatively, additional components can be formulated for timed release of the components when a composition of the disclosure is in contact with a tissue. For instance, components can be formulated in the form of resorbable compositions, such as beads made from alginate, collagen or dextran, glass, or silicate. When a polymer membrane further comprises additional components that can influence the performance of the composition, the components can be therapeutic concentrations of inorganic elements.

Non-limiting examples of additional components that can influence the performance of a composition of the disclosure when a composition is in contact with a tissue include hyaluronic acid, heparin, chondroitin sulfate, keratin sulfate, dermatan sulfate, inorganic elements, and combinations thereof.

Other modifications of a polymer membrane that can influence the performance of the membrane include the architecture of the membrane. For instance, the membrane can be fenestrated in an advantageous pattern to increase cellular communication and diffusion of nutrients and gases. Additionally or alternatively, a polymer membrane can comprise more than one layer of polymer. When a polymer membrane comprises more than one layer of polymer, each layer can exhibit a different physical characteristic. For instance, each layer of the matrix can have different flexibility and/or resorbability. Physical characteristics can be tailored for an intended use of a composition, an intended residence time, and intended release profile of inorganic elements from a composition, and combinations thereof.

When a polymer membrane comprises more than one layer of polymer, a polymer membrane can comprise more than one polymer layer wherein each polymer layer exhibits a different rate of dissolution or tissue residence time. Alternatively or concurrently, a polymer membrane can comprise more than one polymer layer wherein one or more of the layers further comprises components having desirable therapeutic characteristics for use with the composition. A polymer membrane can also comprise more than one polymer layer exhibiting different rates of dissolution wherein one or more layers comprises therapeutic components.

(c) Matrix Composition

As described above, a matrix composition of the disclosure comprises a polymer membrane and bioactive glass associated with the polymer membrane. A composition of the disclosure can be resorbable and flexible.

Physical characteristics of a resorbable flexible matrix can be adjusted to desired treatment parameters of the defect. Physical characteristics of a matrix can be adjusted by adjusting physical characteristics of the polymer membrane of the matrix, physical characteristics of the bioactive glass of the matrix, the arrangement of the bioactive glass relative to the polymer membrane, and combinations thereof. For instance, bioactive glass can be associated with the polymer membrane on a single side of the polymer membrane, on both sides of the polymer membrane, or combinations thereof. Additionally or concurrently, a composition can comprise multiple alternating layers of polymer membrane and/or bioactive glass. Further, a composition can comprise more than one polymer layer, each layer independently having different physical characteristics. Additionally, a composition can comprise more than one bioactive glass composition, wherein each bioglass composition independently has different physical characteristics.

By adjusting physical characteristics of the composition, a matrix composition can be tuned to optimize treatment of a defect in tissue. For instance, when the injury is segmental bone loss, a composition can comprise a polymer membrane comprising an internal polymer layer and at least one polymer layer in contact with each surface of the internal polymer layer, wherein the internal polymer layer further comprises a therapeutic concentration of the inorganic element embedded within the internal polymer layer, and wherein the bioactive glass is associated with at least one surface of the polymer membrane. Such an arrangement allows for a first release of inorganic elements from bioactive glass on the first side of the membrane to induce vascularization of newly formed tissue, followed by a delayed release of inorganic elements from the central layer to induce differentiation and maturation of the newly formed tissue.

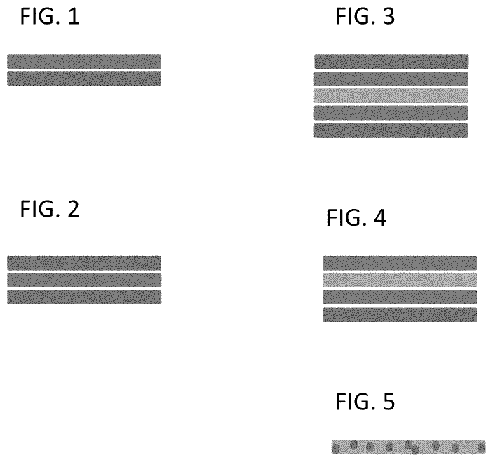

In some aspects, a composition comprises a polymer membrane comprising a single polymer layer associated with bioactive glass on a first external surface of the membrane (FIG. 1). In other aspects, a composition comprises a polymer membrane comprising a single polymer layer associated with bioactive glass on the first and second surfaces of the membrane layer (FIG. 2). In yet other aspects, a composition comprises a polymer membrane comprising more than one polymer layer, and bioactive glass associated with the first or both surfaces of the membrane (FIGS. 3-4). In some aspects, a composition comprises a polymer membrane comprising an internal polymer layer and at least one polymer layer in contact with each surface of the internal polymer layer, wherein the internal polymer layer further comprises a therapeutic concentration of the inorganic element embedded within the internal polymer layer, and wherein the bioactive glass is associated with at least one surface of the polymer membrane.

A composition can further comprise other biomaterial that can enhance treatment of a tissue. When a composition further comprises other biomaterial, the biomaterial can be associated with the matrix, can be a separate component used with the matrix during performance of the procedure, or combinations thereof. For instance, biomaterial can be enclosed within an enclosure formed by the matrix around the volumetric tissue loss. Biomaterial that can be suitable for a composition can and will vary depending on the intended tissue to be repaired, the intended procedure used for repairing volumetric tissue loss, and the membrane composition with which the bioactive glass is associated, among other factors, and can be determined experimentally using methods recognized in the art.

When volumetric tissue loss of a defect is segmental bone loss, compositions can further comprise bone graft material. Non-limiting examples of bone graft material include demineralized bone membrane (DBM), DBM cortical powder, crushed cancellous bone, platelets, platelet lysate, platelet rich plasma, bone marrow aspirate, chondrogenic cells, bioglass, a growth factor, a collagen such as a type I collagen or a type II collagen, or any combination thereof.

When volumetric tissue loss of a defect is muscular tissue loss, compositions can further comprise muscle graft material such as viable muscle tissue, lipoaspirate, microvascular fragments, and combinations thereof.

### II. Method of Using

Another aspect of the disclosure comprises a method of treating a defect in tissue demonstrating volumetric tissue loss. The method comprises obtaining a matrix composition of the disclosure, contacting healthy tissue neighboring the tissue loss with the matrix, and surrounding the defect with the matrix thereby forming an enclosure around the volumetric loss of the defect. The enclosure around the volumetric loss can be in the form of the lost tissue for guiding the repair of the defect in the tissue. Matrix compositions can be as described above in Section I.

Advantageously, using a method of the disclosure allows for localized delivery of inorganic elements to the defective tissue site, resulting in the predictable recruitment of endothelial progenitor cells supporting rapid vasculogenesis and restoration of newly formed basement matrix, ultimately guiding tissue and organ formation. Further, localized delivery of inorganic elements obviates the need to achieve therapeutic serum concentrations of inorganic elements, thereby avoiding potential adverse effects. Use of the matrix of the disclosure also results in the recruitment of mesenchymal progenitor cells from surrounding tissue, which can participate in the biologic processes required to heal the tissue demonstrating volumetric loss. Additionally, antimicrobial properties of bioactive glass of the matrix compositions obviate the need for use of antibiotics normally used during tissue restoration procedures such as the Masquelet technique.

A method of the disclosure can further comprise obtaining biomaterial for treating the defect. The biomaterial can be contained within the enclosure formed by the matrix composition around the defect. Biomaterial suitable for use with a matrix composition can be as described above in Section I(c).

In some aspects a method comprises treating a bone defect in tissue demonstrating volumetric tissue loss. The defect can be segmental bone loss, and the matrix can be from a periosteal replacement forming an enclosure around the segmental bone loss. Periosteum is a dense multilayered and highly vascularized connective tissue envelope that fully encases cortical bone. The thick outer fibrous layer comprises a rich blood vessel network and dense collagen membrane, whereas the thin inner cambium layer contains progenitor cells exhibiting chondrogenic and osteogenic differentiation potential. Each of these layers is recognized to produce paracrine and autocrine mediators displaying osteoinductive and angiogenic activity of critical importance to bone treatment. While not wishing to be bound by theory, it is believed that by providing a periosteal replacement, the method can recruit endothelial cells supporting rapid vasculogenesis. Additional recruitment of mesenchymal progenitor cells from the skeletal muscle participates in the biologic processes of endochondral ossification—the natural mechanism by which long bones are formed during embryogenesis.

Treating a bone defect can comprise obtaining bone graft material and containing the bone graft material within the periosteal replacement. The bone graft material can comprise patient derived bone marrow aspirate concentrate. When a composition further comprises bioactive material, the bone graft material can release hyaluronic acid, further contributing to accelerated vasculogenesis and endochondral ossification promoted by the cell-free matrix comprising resorbable polymer and bioactive glass.

Significantly, a method of treating a bone defect demonstrating volumetric tissue loss significantly and effectively limits the severity of the Masquelet technique normally used to repair a bone defect in tissue demonstrating volumetric tissue loss, by limiting surgical intervention to a single effective procedure, and obviating the need for administration of antibiotics during the Masquelet surgical procedure. Further, this approach effectively reduces or eliminates surgeon's reliance on the harvest of iliac crest bone graft to promote bone healing.

In other aspects a method comprises treating volumetric soft tissue loss, including muscle loss and ruptured tendons and ligaments. When a method of the disclosure comprises treating soft tissue loss, a method comprises using a matrix composition to envelop the tissue loss and form an enclosure around the soft tissue loss in the form of the lost soft tissue.

### III. Method of Manufacturing

Another aspect of the disclosure comprises a method of manufacturing a matrix composition for treating a defect in tissue demonstrating volumetric tissue loss. A matrix composition can be as described in Section I herein. The method comprises obtaining the bioactive glass and the polymer membrane, and associating the bioactive glass with a layer of polymer membrane, thereby forming the matrix.

As described in Section I above, the polymer membrane can comprise more than one layer of polymer membrane, and can further comprise additional components that can influence the performance of a composition of the disclosure. As such, a method of manufacturing the matrix composition can include associating the polymeric layers together to form the multilayered polymer membrane.

Further, a polymer membrane can further comprise components having desirable therapeutic characteristics for use with the composition. The components could be embedded in the polymer membrane during manufacture of the membranes. Alternatively, the components could be adhered to the surfaces of the membrane after manufacture of the membrane.

### Definitions

As used herein, the term “biocompatible” refers to the ability (e.g., of a composition or material) to perform with an appropriate host response in a specific application, or at least to perform without having a toxic or otherwise deleterious effect on a biological system of the host, locally or systemically.

As used herein, the terms “resorbable” and “bioresorbable” refer to the capability of a material to be broken down over a period of time and assimilated into the biological environment. Resorbable and bioresorbable, in the context of an animal body environment, implies that the material is broken down over a period of time and assimilated into the body under normal physiological conditions.

As used herein, the term “flexible” refers to the property of being pliable, able to be compressed, shaped, and manipulated by force of hand, while maintaining integrity, homogeneity of the composition, physical properties, and performance properties.

### EXAMPLES

The publications discussed above are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### INTRODUCTION

### Vasculogenesis and Angiogenesis: Critical Steps to Tissue Development and Repair.

Vasculogenesis identifies the biologic process of differentiation undertaken by mesenchymal cells to form new blood vessels and involves three distinct stages: 1) differentiation of mesodermal cells into angioblasts or hemangioblasts; 2) differentiation of angioblasts or hemangioblasts into endothelial cells; 3) the organization of new endothelium into primary capillary tubules. By contrast, angiogenesis refers to the biologic process whereby formation of new capillary blood vessels occurs through sprouting of pre-existing vessels. Vasculogenesis is typically restricted to embryonic tissue development, while angiogenesis can occur from pre-existing vessels or endothelial precursor cells, participating in embryogenesis as well as normal and pathological vessel formation in adult life.

A primary principle of tissue engineering is the design and application of bioactive, conductive matrices to guide functional assembly of tissues in need of repair. Integration of such matrices within the host largely depends on the design and optimization of scaffold materials promoting local angiogenesis in vivo. Whereas most approaches to date depend on the delivery of biomaterials pre-seeded ex vivo to ensure capillary formation at time of implantation, the ultimate goal would be to achieve vascularization through the design of a cell-free and protein growth factor-free biomaterial exhibiting the unique ability to recruit and activate cellular elements that guide angiogenesis in situ, through the dissolution of factors directly affecting angiogenesis. Protein factors known to influence angiogenesis have been incorporated directly within such matrices. Examples of such factors in current clinical use include platelet derived growth factor (Augment, Wright Medical) and bone morphogenetic protein-2 (Infuse, Medtronic). However, this approach is expensive and can be unpredictable with respect to maintaining protein bioactivity post-sterilization.

Investigators have begun to explore other approaches to induce localized angiogenesis through delivery of inorganic elements. One example is copper containing tripeptide, first discovered to be a component of saliva, human plasma and urine. This bioactive copper peptide declines rapidly as a function of age in humans and coincides with the decrease in regenerative capacity of mammals. Exogenous administration, either topically or systemically, is reported to promote wound healing in addition to the regeneration of hair and damaged skin. Copper alone is recognized to support replicative vitality of fibroblasts from marrow following anticancer radiation therapy. Therefore, copper appears to be an important element facilitating tissue healing. Other elements thought to contribute to tissue repair include silicate, zinc, selenium and cobalt. Each of these inorganic elements serves as a critical cofactor stabilizing enzyme or vitamin secondary structure and function. Lithium is another example of an inorganic element believed to have direct gene targeting through inhibition of specific enzyme activity, the details of which are presented below.

There is a paucity of data supporting direct influence of inorganic elements on mammalian gene expression. Cobalt is a hypoxia mimicking agent recognized to activate hypoxia inducible factor-1 (HIF-1) in mesenchymal stem cells and subsequently to activate HIF-alpha target genes, including VEGF, EPO, BMP, RUNX2.

### WNT Signaling in Embryonic Tissue Development and Repair.

LPR5-independent activation of WNT3a signaling as a strategy to enhance vascularization and repair of different connective tissues, and more specifically bone regeneration, has been demonstrated through exogenous administration of recombinant human WNT protein. While this approach can in time be proven safe and effective through human clinical trials, the regulatory path is burdensome with respect to time and cost. Alternatively, pharmacologic manipulation of the canonical WNT signaling pathway is made possible through exogenous treatment with ionic lithium, an inhibitor of glycogen synthase kinase 3β (GSK3). Inhibition of GSK3 is reported to enhance β-catenin nuclear translocation and downstream WNT signaling. Lithium delivered systemically, as reported for doped drinking water or gavage feeding of animals, was shown to enhance bone strength, accelerate fracture repair, and to restore osteonecrotic bone lesions in aged mice. Tissue repair in these models was reported to occur via enhanced commitment of mesenchymal progenitor cells to those of the osteogenic lineage at the expense of adipogenesis. Therefore, stabilization of β-catenin by localized dissolution of inorganic elements, including lithium, appears to enhance anabolic tissue regeneration, effectively recapitulating those processes known to guide embryonic tissue development. A proposed mechanism of action can comprise →*VEGF-A, BMP-2, -4, -6, →Runx2→WNT signaling.

A more practical approach to achieving targeted restoration of volumetric tissue loss injury is the localized delivery of ionic lithium (Li). Such an approach would obviate the need to achieve therapeutic serum concentrations of Li in the range of 0.5-2 mM, thereby avoiding potential adverse effects on neurological function. Ionic Li can be delivered through the dissolution of bioactive glass (borate or silicate doped with Li) or resorbable polymers containing LiCl₂ such as beads made from collagen, natural polysaccharides (dextran or alginate) or any variety of man-made polymers. Furthermore, it can be advantageous to delay localized release of Li once the newly formed tissue is well vascularized to drive differentiation and maturation. As explained above, newly formed vasculature networks act as a conduit for delivery of pericytes demonstrating multipotent differentiation potential in the formation of muscle, cartilage, bone, tendon, or ligament.

### Example 1. Manufacture of Flexible Matrix Containing Bioactive Glass to Guide Tissue Regeneration for Volumetric Tissue Loss

Bioactive glass was prepared using glass melting procedures in which the glass formers described in U.S. Pat. No. 8,337,875 were used to impart desired biodegradability—borate, silicate, and phosphate at 52.95 wt %, 0 wt %, 4.0 wt %. In accordance with U.S. Pat. No. 8,337,875, this material was further doped with Cu and Zn to enhance vasculogenesis. However, to optimize proteoglycan deposition within newly formed basement matrix, the sulfate salts of zinc and copper were utilized. As an alternative approach to enhance vasculogenesis, doping can be achieved using cobalt oxide. Lithium is further added to drive WNT signaling, recapitulation embryonic tissue formation.

A layer of bioactive glass in the form of fibers or porous spheres is laid down on a sterile surface. Successively, polymer-based layers are added to the glass layer using either a solution of the appropriate polymer and allowing the solvent to evaporate, or by application of a layer of molten polymer. A central polymer layer of the layered structure (optional) contains a solution or suspension of lithium in the form of ions or lithium salts encapsulated in bioactive glass, or alternatively resorbable beads formed from alginate, dextran, or collagen/gelatin. A second layer of glass fibers or beads can also be applied to the top surface as shown in FIG. 3. The resorption time of the polymer layers in contact with the bioactive glass layers would be about 25-30 days, while the resorption of the central lithium containing layer would be greater than 30 days promoting release of the Lithium at times greater than 30 days. The resorption times of the degrading polymers—typically polyesters—can be adjusted by molecular weight and/or composition, e.g., the ratio of glycolide/lactide in a poly (lactide-co-glycolide) polymer or lactide/caprolactone in a poly (lactide-co-caprolactone) polymer. The polymer can additionally be applied as an emulsion containing an aqueous phase comprising proteoglycans such as hyaluronic acid, heparin, chondroitin sulfate, keratin sulfate, or dermatan sulfate. Further tunability of tissue residence time can be achieved through the detailed architecture of the monomer subunits comprising the polymer chains. The formed matrix was allowed to air dry (or lyophilized) and fenestrated in an advantageous pattern to increase cellular communication and diffusion of nutrients and gases. Final product is e-beam sterilized.

### Example 2. Treatment of Large Segmental Bone Defects Using a Modified Masquelet Technique

A 62 year old farmer with a history of tobacco use was airlifted to the ER with an open fracture of his right tibia having experienced a fork lift injury that presented as an 8 cm central diaphyseal defect. After tissue debridement and copious flushing of bone and surrounding soft tissue with antibiotics, injured periosteum was excised leaving a bony defect with no practical way to retain exogenous bone graft. Matrix containing bioactive glass was cut to a width of 14 cm (24 cm length) to span the entire defect. Prior to wrapping the tibia in the matrix, the defect was filled with synthetic bone graft hydrated first by mixing patient-derived concentrated bone marrow aspirate taken from the posterior iliac crest (total aspirate voume 220 cc; total volume delivered with graft 16 cc) using the CellPoint Concentrated Bone Marrow Aspirate System (Isto Biologics, Hopkinton, Mass.). Applied bone graft was subsequently wrapped 3 times using the described cell-free matrix and rigid plate fixation achieved. Injured muscle was repaired with suture and directly laid in direct contact with the matrix. The surgeon was confident that the antimicrobial properties of the matrix containing bioactive glass would effectively kill aerobic and anaerobic bacteria that may have been transmitted by the farm equipment causing injury. Patient demonstrated remarkable bony consolidation at postoperative week 6 that had progressed to remodel and provide pain-free ambulation at his month 6 clinical visit. By eliminating the two-step Masquelet procedure, surprisingly the elderly patient returned to limited farming duties at less than 8 months and to full duty at month 11 without complication.

### Example 3. Salvage Reconstruction of the Vastus Medialis and Rectus Femoris Following Volumetric Muscle Loss Injury Involving a Shark Attack

A 28 year old male surfer was attacked off the northern California coast by a shark resulting in massive laceration injury to his right anterior lower extremities. Approximately 30% of the Rectus femoris and 25% of the Vastus medialis were lost in the attack together with nerve and vascular support tissue. The femur remained otherwise intact. Patient was stabilized within 24 hrs after admission at which time an attempt was made to salvage the limb and restore limited function in this otherwise healthy young male. Viable muscle tissue was harvested post debridement and packed into a dual surface bioactive glass matrix containing time-released lithium and cobalt to enhance revascularization of injured tissue. Two individual matrices were created to provide a template in the general shape of each muscle group. Muscle was finely diced to promote satellite cell outgrowth and combined in situ with autologous fat graft harvested by liposuction. Lipoaspirate was further processed through limited collagenase digestion to yield 50 cc of microvascular fragments to be mixed and loaded into each matrix. The mixed construct filled each of the matrices, which were consequently anchored to existing muscle, ensuring significant overlap of the matrix with otherwise healthy muscle. Skin grafting was delayed until evidence of reperfusion of the muscle was obtained. 7 days post-op reperfusion was initially observed via laser-assisted indocyanine green dye imaging (Novodaq SPY Elite, Stryker). Skin grafting was completed at day 21 without complication. MRI assessment of tissue viability and organization showed remarkable patency and vascular architecture 3 months following muscle repair. Patient initiated physical therapy at 8 weeks and shows continuous improvement in strength and muscle tone with utilization of the injured limb 6 months after initial surgery.

1. A matrix composition for treating a defect in tissue demonstrating volumetric tissue loss, the matrix comprising:
a. a polymer membrane comprising a first surface and a second surface; and
b. bioactive glass associated with a surface of the polymer membrane, wherein the bioactive glass comprises an inorganic element capable of facilitating tissue healing.

2. The composition of claim 1, wherein the matrix is biocompatible, flexible, resorbable, or combinations thereof.

3. The composition of claim 1, wherein the bioactive glass is in the form of fibers or spheres.

4. The composition of claim 1, wherein the bioactive glass is porous.

5. The composition of claim 1, wherein the bioactive glass is borate glass comprising about 50-55 wt % borate, about 0% silicate, and about 3.0-5.0% wt phosphate.

6. The composition of claim 1, wherein the inorganic element is selected from Cu, Se, Co, Zn, Li, and combinations thereof.

7. The composition of claim 1, wherein the inorganic element is Cu, Zn, and Li, Cu and Li, or Co and Li.

8. The composition of claim 1, wherein the bioactive glass is associated with the first surface of the polymer membrane.

9. The composition of claim 1, wherein the polymer membrane further comprises a therapeutic concentration of the inorganic element embedded within the polymer membrane.

10. The composition of claim 9, wherein the inorganic element embedded within the polymer membrane is formulated in the form of beads made from alginate, collagen or dextran, glass, or silicate.

11. The composition of claim 1, wherein the polymer membrane comprises an internal polymer layer and at least one polymer layer in contact with each surface of the internal polymer layer, wherein the internal polymer layer further comprises a therapeutic concentration of the inorganic element embedded within the internal polymer layer, and wherein the bioactive glass is associated with at least one surface of the polymer membrane.

12. A method of treating a defect in tissue demonstrating volumetric tissue loss, the method comprising:
a. obtaining a matrix of claim 1;
b. contacting healthy tissue neighboring the tissue loss with the matrix; and
c. surrounding the defect with the matrix thereby forming an enclosure around the tissue loss.

13. The method of claim 12, wherein the tissue loss is segmental bone loss.

14. The method of claim 13, wherein the method further comprises containing bone graft material within the enclosure.

15. The method of claim 14, wherein the bone graft material comprises patient derived bone marrow aspirate concentrate.

16. The method of claim 12, wherein the volumetric tissue loss is muscular tissue loss.

17. The method of claim 16, wherein the method further comprises containing muscle graft material within the enclosure.

18. The method of claim 17, wherein the muscle graft material comprises viable muscle tissue, lipoaspirate, and microvascular fragments.

19. The method of claim 12, wherein the defect is connective tissue loss.

20. A method of manufacturing a matrix of claim 1, the method comprising:
a. obtaining the bioactive glass;
b. obtaining a polymer membrane; and
c. associating the bioactive glass with a layer of polymer membrane, thereby forming the matrix.
